(19) United States
(12) Patent Application Publication
(10) Pub. No.: US 2021/0298642 A1
(43) Pub. Date: Sep. 30, 2021

(54) SYSTEM AND METHOD FOR TREATMENT OF LOWER BACK PAIN BASED ON BIOMETRICALLY DETERMINED CHANGE IN GAIT

(71) Applicant: Click Therapeutics, Inc., New York, NY (US)

(72) Inventors: Caroline Pena, New York, NY (US); Allison Hirsch, Brooklyn, NY (US)

(73) Assignee: Click Therapeutics, Inc., New York, NY (US)

(21) Appl. No.: 17/213,089

(22) Filed: Mar. 25, 2021

Related U.S. Application Data
(60) Provisional application No. 62/994,815, filed on Mar. 25, 2020.

(51) Int. Cl. A61B 5/11; A61B 5/00

(52) U.S. Cl. CPC A61B 5/112; A61B 5/7246; A61B 5/1128; A61B 2562/0223; A61B 5/6898; A61B 5/4824; A61B 2562/0219; A61B 5/4848

(57) ABSTRACT

A system for measuring and assessing biometrically determined changes in gait is provided. The system includes a processor, a memory, and instructions written on the memory, wherein the instructions when executed by the processor cause the system to: acquire a baseline gait pattern, acquire a subsequent gait pattern; compare the baseline gait pattern to a subsequent gait pattern, interpolate the baseline gait pattern and the subsequent gait pattern, validate the baseline gait pattern and the subsequent gait pattern, correlate the baseline gait pattern and the subsequent gait pattern; update the baseline gait pattern, predict a likelihood of a flare-up, and communicate the likelihood of a flare-up to a user.

### CLAIM OF PRIORITY

This application claims priority from U.S. Provisional Patent Application No. 62/994,815, filed on Mar. 25, 2020, the contents of which are incorporated herein by reference.

### FIELD OF THE INVENTION

This disclosure relates to a personalized digital therapeutic invention for chronic lower back pain. Specifically, this disclosure relates to systems and methods for measuring and assessing biometrically determined changes in gait in order to improve the effectiveness of therapeutic treatment of chronic lower back pain as well as to predict the likelihood of flare-ups.

### INTRODUCTION

The clinical practice guidelines for the treatment of chronic lower back pain recommend that clinicians advise patients on self-care. The treatment of chronic low back pain may be challenging due to the fact that patients may cycle through periods of greater and lesser amounts of pain, as well as map onto inconsistent self-care practices. Additionally, when patients feel better, they may engage in a greater amount of activity than previously, which may result in subsequent increased rates of pain.

Self-care may be challenging for patients who are unsure how to vary self-care practices as states of pain fluctuate. Accordingly, there is a need for a system and method to enable individuals to accurately monitor and improve therapeutic treatment of chronic lower back pain. This disclosure includes systems and methods of personalized therapeutic intervention for the treatment of chronic lower back pain which may improve the therapeutic treatment of lower back pain through the repeated measurement and assessment of biometrically determined changes in gait through the use of a mobile device such as a smartphone.

### BRIEF DESCRIPTION OF THE DRAWINGS

The following drawings are intended to serve as illustrative embodiments of the features disclosed in the present disclosure.

FIG. 1 is a diagram illustrating a system for measuring and assessing biometrically determined changes in gait in order to improve the effectiveness of therapeutic treatment of chronic lower back pain as well as predict the likelihood of flare-ups, in accordance with the present disclosure;

FIG. 2 is flow-chart illustrating a method for measuring and assessing biometrically determined changes in gait in order to improve the therapeutic treatment of chronic lower back pain and predicting the likelihood of flare-ups in chronic lower back pain, in accordance with the present disclosure; and

FIG. 3 is an additional flow-chart illustrating another exemplary method for measuring and assessing biometrically determined changes in gait in order to improve the therapeutic treatment of chronic lower back pain and predicting the likelihood of flare-ups in chronic lower back pain, in accordance with the present disclosure.

FIG. 4 illustrates a main workflow incorporating one or more algorithms.

FIG. 5 illustrates a process of getting data from a database.

FIG. 6 illustrates a process of searching matches between two lists.

FIG. 7 illustrates a process of splitting a global dataset to train and test datasets.

FIG. 8 depicts a diagram of a neural network.

FIG. 9 illustrates a computer code configured to execute one or more algorithms.

FIGS. 10A-B depict an example of predicted pain points via a linear regression method.

FIGS. 11A-C depict tables of printed datasets, normalized data, and coefficients of parameters.

### DETAILED DESCRIPTION OF THE INVENTION

The description of illustrative embodiments according to principles of several illustrative embodiments is intended to be read in connection with the accompanying drawings, which are to be considered part of the entire written description. In the description of embodiments disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits are illustrated by reference to certain exemplified embodiments and may not apply to all embodiments.

Accordingly, the invention expressly should not be limited to such illustrative embodiments illustrating some possible non-limiting combination of features that may exist alone or in other combinations of features; the scope of the claimed invention being defined by the claims appended hereto.

This disclosure describes the best mode or modes of practicing the invention as presently contemplated. This description is not intended to be understood in a limiting sense, but provides an example of the invention presented solely for illustrative purposes by reference to the accompanying drawings to advise one of ordinary skill in the art of the advantages and construction of the invention. In the various views of the drawings, like reference characters designate like or similar parts.

The various embodiments described herein describe a system and method relating to a personalized digital therapeutic invention for chronic lower back pain based on biometrically determined changes in gait.

Human gait refers to locomotion achieved through the movement of human limbs. Specifically, human gait is defined as the bipedal, biphasic forward propulsion of center of gravity of the human body, in which there are alternate sinuous movements of different segments of the body with the least expenditure of energy. Different gait patterns are characterized by differences in limb-movement patterns, overall velocity, forces, kinetic and potential energy cycles, and changes in the contact with the surface (i.e., ground, floor, etc.). Human gaits are the various ways in which a human can move, either naturally or as a result of specialized training.

Human gaits may be classified in various ways. Every gait may be generally categorized as either natural (i.e., one that humans use instinctively) or trained (i.e., a non-instinctive gait learned via training). Examples of the latter include hand walking and specialized gaits used in martial arts. Gaits can also be categorized according to whether the person remains in continuous contact with the ground.

Chronic lower back pain (“CLBP”) can have its origin within the gait cycle and lower extremity (“LE”) function in particular. Considering that individuals will take between 5-10,000 steps per side per day on average, having dysfunction within the lower extremity can produce a repetitive strain injury (“RSI”) on par with any other repetitious activity known to cause symptoms. While impact shock has been theorized as the traumatic event during gait, other factors may be far more important when considering how the lumbar spine can be stressed during walking.

There are three basic factors present during the various phases of any step that can be repetitively stressful to the lower back. These steps include flexion of the lumbar spine during mid single support phase and iliopsoas activity and lateral trunk bending at toe off. While each of these mechanical dysfunctions can exist as individual entities, it is best to view the events present during walking as a continuum, with one aspect either leading to or perpetuating the other(s).

Accordingly, a person's gait is made up of multiple step cycles by each foot, which may be categorized into subevents, which are described below. Step (1) of the step cycle may be categorized as “Initial Contact,” during which the first foot makes initial contact with the ground. Step (2) of the step cycle may be categorized as “Loading Response,” in which the majority of a person's body weight is transferred to the first foot. Step (3) may be categorized as “Midstance,” in which the swinging-foot (opposite foot) passes the first foot. Step (4) may be categorized as “Terminal Stance,” in which the first foot loses contact with the ground. Steps (5), (6), and (7) may be categorized as “Preswing,” “Initial Swing,” and “Midswing,” respectively, during which the opposite foot makes initial contact with the ground, majority of a person's body weight is transferred to the foot, and the foot loses contact with the ground. Step (8) may be categorized as “Terminal Swing,” in which the first foot makes contact with the ground once again. While every person goes through these step cycles, gait pattern has been found to be unique on a person-to-person basis, due to differences and varying weights of importance in these subevents, inconsistencies across step cycles, and variable conditions such as walking speed.

CLBP has been shown to cause difficulties walking and may influence a patient's gait. Since gait is made up of subevents, changes in a patient's gait may equate to change in these subevents described above. The present disclosure includes a system and method for the therapeutic treatment of CLBP through the use of a mobile device, which may include an inertial measurement unit, (“IMU”), which may include a combination of accelerometers, gyroscopes and magnetometers, Global Positioning System (GPS), and other electronic components, and may be used analyze the characteristics of a patient's gait, such as duration, distance, gait speed and cadence, or step speed. While these characteristics can be used to monitor changes in gait in a controlled lab setting, they can be unreliable in a patient's daily life as gait speed can be situationally influenced (e.g., person walking on a crowded street, person on a commuter train). Monitoring changes in a patient's unique gait pattern with a speed-independent algorithm may be able to detect improvements of therapeutic treatments and predict flare ups of CLBP.

Gait detection technology may be used to capture gait at a single point in time. The present disclosure also includes using gait detection technology for the repeated measurement of an individual's gait, starting with a baseline measurement. Subsequent measurements may be collected as well as compared to baseline measurements, and other post-baseline measurements, in order to determine changes in gait across time. Furthermore, gait measurements relative to each other may be used in order to be gait difference scores. Accordingly, gait difference scores may be categorized in a number of different categories, described in greater detail hereinbelow.

Different mechanisms of action (e.g., physical therapy) and product features (e.g., personalized motivational messages) may be provided to individual users based on their gait change difference score. Further performance distinctions may be made in addition to categorization. Within each category, the difference scores may be categorized along a continuum ranging from worst to best. Mechanisms of action (“MOAs”) (e.g., physical therapy) will be provided to users with varying levels of intensity, appropriate to the individual's level of functioning, as determined by the difference score. This present disclosure includes a system and method wherein individual gait is constantly being reevaluated, throughout the duration of the therapeutic treatment.

In order to be sensitive to changes in gait that occur post-baseline, gait difference scores may also be calculated using post-baseline data. This may be done in order to detect subtle changes which may be indicative of a flare-up. Psychosocial interventions aimed at developing coping strategies and managing flare experiences may be more effective if provided to users when they need them most, i.e., when changes in their movement suggests an upcoming flare episode.

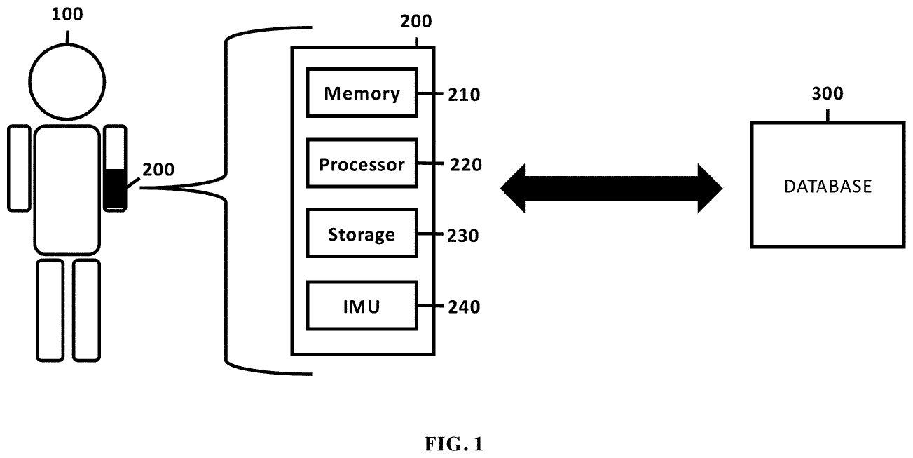

With reference to FIG. 1, a diagram illustrating an illustrative system for measuring and assessing biometrically determined changes in gait in order to improve the effectiveness of therapeutic treatment of chronic lower back pain as well as predict the likelihood of flare-ups is shown in accordance with the present disclosure. With continued reference to FIG. 1, the system may include, but is not limited to: an individual 100, a mobile device 200 (e.g., a smartphone), and a database 300. With reference to FIG. 1, the mobile device 200 may be configurable to measure and assess biometrically determined changes in gait in order to improve the effectiveness of therapeutic treatment of chronic lower back pain as well as predict the likelihood of flare-ups. With continued reference to FIG. 1, the mobile device 200 device may be coupled to or may be within a functional proximity of the individual 100. The functional proximity may depend on the specific electronic components comprising the electronic device.

The mobile device 200 may include, but is not limited to: a memory 210, a processor 220, a storage 230, and an inertial measurement unit 240, (“IMU”), which may include a combination of accelerometers, gyroscopes, magnetometers, GPS, and other electronic components, may be used analyze characteristics of a patient's gait, including but not limited to, duration, distance, gait speed and cadence, or step speed. The IMU may function to obtain any biometric data related to gait. The mobile device may also include a display screen. The mobile device may also include a plurality of additional sensors, described in greater detail below. While the present disclosure discusses the use of an IMU included within a mobile device, it is to be understood by one of skill in the art that the same functionality may be achieved through the use of individual or a plurality of sensors not contained within a single IMU, and may either be coupled to each other or connected wirelessly.

The IMU 240 may include an accelerometer. An accelerometer may measure gravitational pull to determine the angle at which a device is tilted with respect to the Earth. By sensing the amount of acceleration, a user may be able to analyze how a device is moving. In accordance with the present disclosure, the accelerometer may be able to detect how a person's limbs are moving, i.e., gait and step cycles. For example, an accelerometer may be used to determine, for example, whether a body or object is moving uphill, whether a body or object will fall over if it tilts any more, or whether a body or object is traveling horizontally or angling downward. An accelerometer may also be used to obtain a better understanding of the surroundings of a body. In accordance with the present disclosure, the accelerometer may be configured to obtain gait data.

An accelerometer may be comprised of a multitude of different components and may function in a multitude of different ways. For example, a piezoelectric effect accelerometer uses microscopic crystal structures that become stressed due to accelerative forces. Accordingly, these crystals create a voltage from the stress, and the accelerometer interprets the voltage to determine velocity and orientation. As another example, a capacitance accelerometer senses changes in capacitance between microstructures located next to the device. If an accelerative force moves one of these structures, the capacitance will change, and the accelerometer will translate that capacitance to voltage for interpretation.

Accelerometers may be made up of many different components, and may already be integrated into existing mobile devices such as smartphones. These components may be integrated into the main technology and accessed using the governing software or operating system. In the present disclosure, embodiments of the present invention may include an accelerometer disposed within the mobile device. However, it is contemplated in this present disclosure that the accelerometer may also be disposed outside of the mobile device, without departing from the scope and spirit of the present invention.

Typical accelerometers may be comprised of multiple axes. Two axes may be used to determine most two-dimensional movement, while a third axis may be used to detect 3D positioning. Mobile devices may make use of three-axis models. Accelerometers in mobile devices may be sensitive enough to measure very minute shifts in acceleration. Accordingly, the more sensitive the accelerometer, the more easily it can measure acceleration. Embodiments of the present invention may use a three-axis accelerometer to measure gait. It is contemplated in the present disclosure that an accelerometer with more or less axes may be used to accomplish the same function without departing from the scope and spirit of the invention.

Other types of motion detection sensor devices may include the use of at least one: passive infrared (“PIR”) sensor, microwave sensor, ultrasonic sensor, tomographic motion detector, gesture detector, and the like. Furthermore, motion detection may also be accomplished through the use of a video, wherein the output of a video camera may be used as an input to detect motion. It is contemplated in the present disclosure that any type of accelerometer known to one of ordinary skill in the art may be used in order to measure gait, without departing from the scope and the spirit of the present invention. Furthermore, it is contemplated that a combination of multiple sensors may be used in order to measure gait. Accordingly, the use of multiple sensing technologies may help reduce false triggering. For example, a dual technology sensor may combine a PIR sensor and a microwave sensor into one unit. For motion to be detected, both sensors must trip together, which in turn may lower the probability of a false alarm. PIR technology may be paired with another sensor model to maximize accuracy and reduce energy use. For example, PIR draws less energy than emissive microwave detection, and so sensor device systems may be calibrated so that when a PIR sensor is tripped, a microwave sensor may be activated.

The IMU 240 may include a gyroscope to assist in measuring and assessing gait. Accordingly, a gyroscope is a device that may be used to measure or maintain orientation and angular velocity. Accordingly, a gyroscope is a spinning wheel or disc in which the axis of rotation (spin axis) is free to assume any orientation by itself. The gyroscope maintains its level of effectiveness by being able to measure the rate of rotation around a particular axis. Using the key principles of angular momentum, the gyroscope helps indicate orientation. In accordance with the present disclosure, embodiments of the invention may include the use of a gyroscope in order to indicate the orientation of human limbs in order to measure and assess gait to increase the effectiveness of therapeutic treatment of chronic lower back pain.

The IMU 240 may include a magnetometer. Accordingly, a magnetometer is a device that measures magnetism. More specifically, a magnetometer may measure the direction, strength, or relative change of a magnetic field at a particular location. For example, a magnetometer may include a compass, which the direction of an ambient magnetic field, in this case, the Earth's magnetic field. In accordance with the present disclosure, the IMU 240 may include a magnetometer which may be configured to obtain gait data.

The IMU 240 may include a GPS sensor. Accordingly, the GPS sensor may be configured to obtain gait data. With further continued reference to FIG. 1, it has been contemplated in the present disclosure that one of ordinary skill in the art would understand that the IMU 240 may include other electronic components.

With continued reference to FIG. 1, the mobile device 200 may include a processor 220 and a memory 210. The processor 220 may be any type of processing device for executing software instructions. The memory 210 may include both a read-only memory (“ROM”) and a random access memory (“RAM”). As will be appreciated by those of ordinary skill in the art, both the ROM and the RAM may store software instructions for execution by the processor 220, described in greater detail below.

The memory 210 may contain instructions, wherein when the instructions are executed by the processor, cause the system measure and assess gait data to increase the effectiveness of therapeutic treatment of CLBP.

The processor 220 and memory 210 may be connected, either directly or indirectly, through a bus or alternate communication structure to one or more peripheral devices. For example, the processor 220 and/or the memory 210 may be directly or indirectly connected to additional memory storage 230, such as the hard disk drive, the removable magnetic disk drive, the optical disk drive, and the flash memory card. The processor 220 may also be directly or indirectly connected to one or more input devices and one or more output devices. The input devices may include, for example, a keyboard, a touch screen, a remote control pad, a pointing device (i.e., mouse, touchpad, stylus, trackball, joystick, etc.), a scanner, a camera, and/or a microphone. The output devices may include, for example, a monitor, haptic feedback device, television, printer, stereo, and/or speakers.

The database 300 may include the biometric data related to individual gait, data relating to the gait of the general population, data relating to the gait of a particular population such as the gait of a demographic group such as a particular age, sex, race, weight, and the like, or any other data obtained that may be used to increase the effectiveness of therapeutic treatment and predict the likelihood of CLBP flare-ups.

Furthermore, the mobile device 200 may be directly or indirectly connected to one or more network interfaces for communicating with a database 300. This type of network interface, which may also be referred to as a network adapter or network interface card (“NIC”), may translate data and control signals from the computing unit into network messages according to one or more communication protocols. The communication protocols may include, but are not limited to, Transmission Control Protocol (“TCP”), the Internet Protocol (“IP”), and/or User Datagram Protocol (“UDP”). An interface may employ any suitable connection agent for connecting to a network, including but not limited to, a wireless transceiver, a power line adapter, a modem, and/or an Ethernet connection.

Furthermore, in addition to the input, output, and storage peripheral devices specifically described hereinabove, the computing device may be connected to a variety of other peripheral devices, including some that may perform input, output, and storage functions, or some combination thereof.

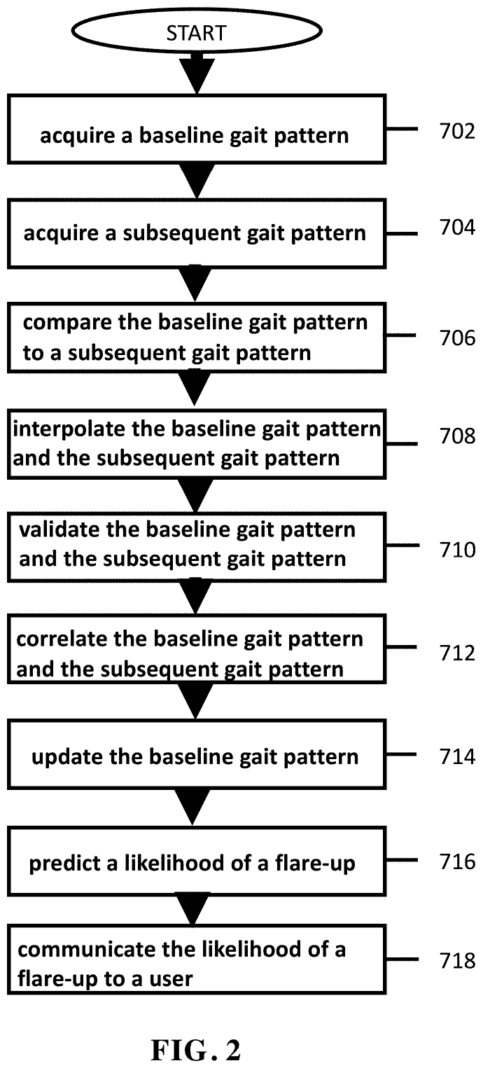

With reference to FIG. 2, a flowchart illustrating an illustrative embodiment of a method for measuring and assessing biometrically determined changes in gait in order to improve the effectiveness of therapeutic treatment of chronic lower back pain as well as predict the likelihood of flare-ups is shown in accordance with the present disclosure. With continued reference to FIG. 2, a method for detecting changes in a patient's gait caused by CLBP flareups utilizing a mobile device-based algorithm is included in the present disclosure. Accordingly, the algorithm may create a baseline gait pattern profile at multiple walking speeds for a user using the inertial measurement unit in their smartphone. As a patient walks throughout their day, the algorithm may calculate gait patterns for a user for multiple (e.g., three) walking periods and compare the calculated gait pattern to the baseline gait pattern. To account for variable walking speeds, the algorithm may incorporate an interpolation method, in order to interpolate the calculated gait pattern or baseline gait pattern. Differences between the interpolated calculated and baseline gait patterns may be determined through correlation calculations. The algorithm may be adaptive wherein the algorithm may update the calculated and interpolated stored gait patterns to reflect any trends.

In accordance with the present disclosure, various interpolation methods may be used in order to interpolate a gait pattern. Accordingly, interpolation methods that may be used to interpolate a gait pattern may include, but are not limited to, linear interpolation and polynomial spline interpolations. Linear splines are linear functions. Polynomial splines may include cubic, quadratic, and other higher order functions. Cubic and quadratic spline interpolation may be preferred when applied to real world data, wherein cubic and quadratic spline interpolations may provide smooth curve fitting.

The initial gait measurement may be taken using a mobile device (i.e., smartphone, tablet, etc.). More specifically, the initial gait measurement may be taken using at least one sensor or sensor device disposed within the user device. Even more specifically, the at least one sensor or sensor device may include a motion sensor. For example, an accelerometer, an electromechanical device used to measure the acceleration of forces, may be used to detect motion. The specific steps of the algorithm are described in greater detail hereinbelow.

With continued reference to the present disclosure, as a patient walks throughout their day, the algorithm may calculate gait patterns for a user for multiple (e.g., three) walking periods and compare the calculated gait pattern to the baseline gait pattern. To account for variable walking speeds, the algorithm may incorporate an interpolation method, in order to interpolate the calculated gait pattern or baseline gait pattern. Differences between the interpolated calculated and baseline gait patterns may be determined through correlation calculations. The algorithm may be adaptive wherein the algorithm may update the calculated and interpolated stored gait patterns to reflect any trends.

Various illustrative embodiments of the invention described in the present disclosure may be implemented using electronic circuitry configured to perform one or more functions. For example, in an embodiment of the present disclosure, the mobile device may be implemented using one or more application-specific integrated circuits (“ASICs”). More typically, however, components of various illustrative embodiments of the present disclosure may be implemented using a programmable computing device executing firmware or software instructions, or by some combination of purpose-specific electronic circuitry and firmware or software instructions executing on a programmable computing device.

With reference to FIG. 2, a flow-chart illustrating a method for measuring and assessing biometrically determined changes in gait including the use of a Gait Detection Algorithm is shown in accordance with the present disclosure. The implementation of the Gait Detection Algorithm may include, but is not limited to, the following steps: baseline gait pattern acquisition, gait pattern comparison, and baseline gait pattern updates. When patients are first introduced to Gait Pattern Monitoring as a Mission or Feature of the Digital Therapeutic Treatment, they may be asked to walk in a straight line on a flat surface at: 1) normal pace, 2) slow pace, and 3) fast pace for approximately 15 meters. The patient may be asked to hold their phone in an area that is close to their body, such as rear pants pocket, front pants pocket, shirt pocket, and avoid areas where it may move around, such as loose in backpack, messenger bag or purse, as accuracy of recordings rely on devices being close to a person's center of gravity and held in position to avoid excess noise. These initial recordings may be used to capture a patient's unique gait pattern and ranges in their speed. Gait patterns (“G”), extrapolated from these recordings from the inertial measurement unit and GPS capabilities of a patient's mobile device. As the inertial measurement unit (IMU) in mobile devices measures linear and angular motion, the data outputted is acceleration, measured in units meters per second squared (m/s²), and rotational rate, measured in radians per second (rad/s). GPS data collection capabilities of mobile phones gathers latitude and longitude coordinates. As gait pattern is defined in units of acceleration, data may be processed in 3 ways: (1) Utilizing acceleration data from the IMU; (2) Calculating acceleration from recorded GPS coordinates and time stamps; and (3) Determine average acceleration through cross comparison of acceleration data from the IMU and calculated acceleration from GPS coordinate data. G may be defined as the shape of the trace of a function of acceleration, (“a”), and sample number, (“s”):

G(s,a); 0≤s≤N

Where a ranges from [−aₘₐₓ, aₘₐₓ] where aₘₐₓ is the maximum acceleration value for the patient and s ranges from [0, N] where N is the total number of samples recorded in that recording period. Gait patterns may be analyzed based on one full step cycle, beginning when a foot makes initial contact with the ground and ending with that foot and ending with that foot returning to that position, resulting in acceleration of that foot starting at about zero, increasing when in swing and decreasing back to about zero. A step cycle can be isolated from the recordings by finding where in the recording a approaches zero and obtaining the corresponding subset of sample values, s₀ to s_{N}:

G(s,a≈0); s₀≤s≤s_{N}

From this set, two consecutive values for where a approached 0 were extracted, sₙ and sₙ₊₁, and used to define a baseline gait pattern, G₀, from the recording:

G₀(s,a≈0); sₙ≤s≤sₙ₊₁

The duration in terms of sample number or range of the baseline gait pattern, R₀, was also calculated for later reference in gait comparison:

R₀=sₙ₊₁−sₙ

This step cycle extraction was done for the three conditions recorded, resulting in three gait patterns. Gait patterns for slow, G_{0,S}, and fast, G_{0,F} paces were calculated and saved for later validation of the interpolation step in the algorithm. The gait pattern at normal pace, G₀, was set as the patient's baseline gait pattern.

Gait comparisons may include a number of steps, which may include but are not limited to: initial gait pattern comparisons, gait pattern interpolation and validation, and gait pattern correlations. For example, gait patterns may be calculated for a patient three times a day, where the patient walks for about 15 meters. If the patient does not walk for about 15 meters at least once from 7:00 AM to 3:00 PM, they may receive a notification informing them they have not been very active that day and asking them to try taking a walk. The duration of the calculated gait pattern, G_{C}, in terms of sample number, R_{C}, was first calculated and compared to the duration of the baseline gait pattern, R₀. Differences in the duration may reflect a difference in walking speed as sampling rate (samples/second) remains constant with use of the same smartphone. Interpolation may be used to predict data points utilizing existing data points, in order to normalize for speed-based differences in the gait patterns. Initial gait pattern comparisons were done in order to determine which calculated gait pattern (G₀ or G_{C}) would be interpolated. Gait pattern interpolation, G_{0,i} or G_{C,i}, was determined by the following conditions:

$$1. {R}_{0} > {R}_{C} \to {G}_{C , i} , {G}_{0}$$

$$2. {R}_{0} < {R}_{C} \to {G}_{C} , {G}_{0 , i}$$

$$3. {R}_{o} \pm SR \text{/} 2 ≅ {R}_{C} \pm SR \text{/} 2 \to {G}_{C} , {G}_{0}$$

If the value of R₀ was greater than R_{C}, this indicated that G_{C} was at a faster walking speed than G₀ and G_{C} must be interpolated for accurate comparison to be done between the gait patterns. If R_{C} was greater than R₀, this indicated that G₀ was at a faster walking speed than G_{C} and G₀ must be interpolated for accurate comparison to be done between the gait patterns. If R₀ and R_{C} are roughly the same value, within a deviation of half the sampling rate (samples/second) of the smart phone, no interpolation is done and G₀ and G_{C} are directly compared. Through implementing interpolation of both baseline gait pattern and calculated gait pattern, this takes into account situations that may cause the patient to increase their walking speed (running late to work/meeting/appointment, walking in rain/snow/other inclement weather, or emergencies) or decrease their walking speed (walking in a crowd, walking on uneven surfaces, or leisurely walking). Various interpolation methods can be used in order to interpolate one of the gait patterns. These include, but are not limited to, linear interpolation and polynomial spline interpolations. Linear and polynomial spline interpolations fit splines, or specialized piecewise functions, in order to form consecutive data sets. Linear splines are linear functions, while polynomial splines can include cubic, quadratic, and other higher order functions. Cubic and quadratic spline interpolation are often preferred when applied to real world data as they offer smooth curve fitting. After the gait pattern to be interpolated (G_{X}) is identified, it undergoes the interpolation method selected:

$$\begin{matrix}{G}_{X} ( s , a ) ; {s}_{n} \leq s \leq {s}_{n + 1} \\ ↓ \\ {G}_{i} ( s , a ) ; {s}_{m} \leq s \leq {s}_{m + 1}\end{matrix}$$

This results in a new gait pattern, G_{X,i}, with a new range in s that fits the comparison criteria of Rᵢ, which is equal to the difference of the interpolated step cycle threshold sample values sₘ₊₁ and s_{M}, within half of the sampling rate. The accuracy of the interpolation gait pattern is validated through comparison with the slow or quick pace gait patterns recorded during the baseline gait pattern acquisition step. Comparison with slow, G_{0,S}, or fast, G_{0,F}, pace gait patterns depended on whether the calculated gait pattern was interpolated (faster than baseline) or not (slower than baseline). Gait patterns were overlaid and differences in trace shape would be examined. If accuracy is found to be low (<0.9), a different interpolation method may be used used (i.e., linear interpolation, n-th order polynomial interpolation) until adequate accuracy is achieved. Once interpolation step is shown to be consistently accurate (accuracy >=0.9 over 10+ comparisons), this validation step can be omitted.

The couple of gait patterns (G_{0,i} and G_{C}, G₀ and G_{C,i}, or G₀ and G_{C}) are then compared through correlation calculations. Pearson's correlation method may be used to define the linear relationship between two variables or sets of data. The Pearson's correlation coefficient, p_{X,Y}, is defined as:

$${p}_{X , Y} = \frac{A ( X - A ( X ) ( Y - A ( Y ) )}{{σ}_{X} {σ}_{Y}} ; - 1 \leq {p}_{X , Y} \leq 1$$

where X and Y are the two variables or datasets of interest, A is an averaging function, and σ_{X} and σ_{Y} are the standard deviations of X and Y respectively. In order to find the correlation between the interpolated gait pattern, Gᵢ, and recorded gait pattern (G₀ or G_{C}), G_{Z}, the Pearson's correlation coefficient would be defined as:

$${p}_{{G}_{Z} , {G}_{i}} = \frac{A ( {G}_{Z} - A ( {G}_{Z} ) ) ( {G}_{i} - A ( {G}_{i} ) )}{{σ}_{{G}_{Z}} {σ}_{{G}_{i}}} ; - 1 \leq {p}_{{G}_{Z} , {G}_{i}} \leq 1$$

The value of p_{X,Y} approaching −1 indicates a negative linear correlation, p_{X,Y} approaching 1 indicates a positive linear correlation and p_{X,Y} approaching 0 indicates no linear correlation. In terms of correlation between gait patterns, a p_{Gz,Gi} value approaching −1 indicates the two gait patterns have a linearly negative relationship, a p_{Gz,Gi} value approaching 1 indicates the two gait patterns have a linearly positive relationship, and a p_{Gz,Gi} value approaching 0 indicates the two gait patterns have no correlation. As CLBP has been shown to cause difficulties walking and changes in gait over time, considering values of p_{Gz,Gi} ranging from −1 to 0, 0 not inclusive, values that closely approach −1 (e.g., −0.9) indicate considerable worsening (e.g., a change in gait pattern) while values closer to 0 (e.g., −0.01) indicate slight worsening (a flare-up). p_{Gz,Gi} of 0 indicates no change. Considering values of p_{Gz,Gi} ranging from 0 to 1, 0 not inclusive, values that closely approach 1 (e.g., 0.9) indicate substantial improvement while values closer to 0 (e.g., 0.01) indicate no improvement or slight improvement.

As this algorithm is meant to help mediate chronic lower back pain treatment, calculated/interpolated gait patterns may be stored and evaluated for trends on a monthly basis to examine any long-term change to baseline gait pattern. Trends can be evaluated using methods for evaluating trends in time-series data such as, but not limited to, linear trend estimations, non-parametric methods such as Mann-Kendall test, and/or through averaging calculated/interpolated gait patterns and correlation comparison to baseline. If a trend is found in the calculated/interpolated gait patterns that differs from the baseline gait pattern, the baseline gait pattern may be updated to the averaged calculated/interpolated gait patterns.

With continued reference to FIG. 2, at step 702, a baseline gait pattern may be acquired at an initial point in time using a mobile device, as described in greater detail above. With further continued reference to FIG. 2, at step 704, a subsequent gait pattern may be acquired at a subsequent moment in time likewise using a mobile device. At step 706, the baseline gait pattern is compared to the subsequent gait pattern. At step 708, the baseline gait pattern and the subsequent gait pattern is interpolated. At step 710, the baseline gait pattern and the subsequent gait pattern is validated. At step 712, the baseline gait pattern and the subsequent gait pattern is correlated. At step 714, the baseline gait pattern is updated. At step 716, a likelihood that a CLBP flare-up may occur is predicted. At step 718, the likelihood of a flare-up is communicated to a user.

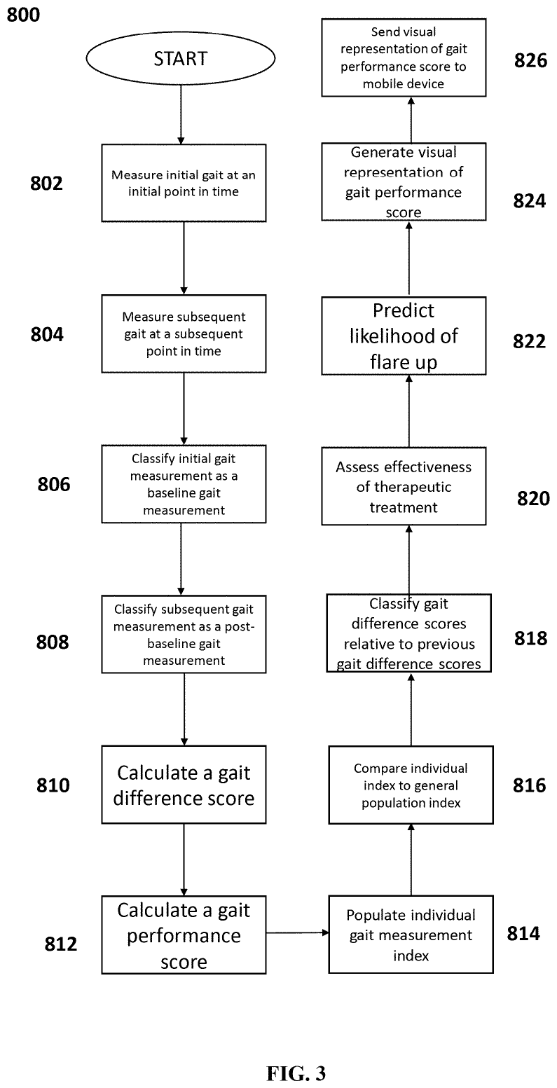

With reference to FIG. 3, another flow-chart illustrating an illustrative method for measuring and assessing biometrically determined changes in gait to improve the therapeutic treatment of and predict the likelihood of flare-ups associated with CLBP. With continued reference to FIG. 3, at step 802, an initial gait measurement may be taken at an initial point in time. The initial gait measurement may be taken using a mobile device (e.g., smartphone, tablet, etc.), as described in greater detail above in the present disclosure. More specifically, the initial gait measurement may be taken using at least one sensor or sensor device disposed within or coupled to the user device. Even more specifically, the at least one sensor or sensor device may include a motion sensor. For example, an accelerometer, an electromechanical device used to measure the acceleration of forces, may be used to detect motion, as well as any other inertial measurement unit. With continued reference to FIG. 3, at step 804, a subsequent gait measurement may be taken at a subsequent moment in time likewise using a mobile device. At step 806, the initial gait measurement may be classified as a baseline gait measurement. At step 808, the subsequent gait measurement may be classified as a post-baseline gait measurement.

With continued reference to FIG. 3, at step 810, a gait difference score may be calculated based on obtaining the difference between the baseline gait measurement and the post-baseline gait measurement. At step 812, a gait performance score may be calculated based on the gait difference score. The gait difference score and the gait performance score may be indicators of whether a treatment is effective in treating lower back pain. Further, the gait difference score and the gait performance score may be continuously measured to track the continuous progression of changes in gait.

With continued reference to FIG. 3, at step 814, an individual gait measurement index, the correlation comparison between the baseline gait pattern and calculated gait pattern, may be populated using at least one initial individual gait measurement, subsequent individual gait measurement, individual gait difference score, and/or individual gait performance score.

At step 816, the individual gait performance score may be compared to a general population gait measurement index or a particular demographic, in order to obtain a relative gait performance score. The general population gait measurement index may be populated using gait measurements from the general population. The general population gait measurement index may include gait measurements in accordance with different classifications. For example, the general population gait measurement index may classify gait measurements, gait difference scores, and gait performance scores, in accordance with the following classifications, including but not limited to: age, height, weight, race, ethnicity, sex, disability, etc. With continued reference to step 816, by comparing individual gait performance scores to the general population gait measurement index, the present method may be able to quantify a disability. Furthermore, classifying a disability may influence the very first mechanisms of action (“MOAs”) presented as well as their properties, including but not limited to: duration, frequency, intensity, etc.

At step 818, the gait performance score is classified in a number of different categories. For example, in one embodiment of the present disclosure, there may exist three categories of progress of therapeutic intervention: 1) “no change”, 2) “improvement”, and 3) “decline.” The “no change” category may indicate that there has been no change in post-baseline gait measurement relative to the baseline gait measurement. The “improvement” category may indicate that there has been some improvement relative to the baseline gait measurement. The “decline” category may indicate decline in gait measurement relative to the baseline gait measurement. It is further contemplated that the gait data and information obtained through this method may be used to predict flare-ups of CLBP, as discussed in greater detail herein this present disclosure.

At step 820, the algorithm assesses the effectiveness of the therapeutic treatment for the CLBP based on the measured and calculated gait data. With continued reference to FIG. 3, at step 822, the algorithm predicts the likelihood of a CLBP related flare-up, likewise based on the measured and calculated gait data.

At step 824 a visual representation of gait performance score may be generated. Furthermore, at step 826, a visual representation of gait performance score may be sent to a mobile device of an individual.

It should be understood by one of ordinary skill in the art that additional steps may be included, steps may be repeated, and/or steps may be omitted without departing from the scope and spirit of the invention. Furthermore, it should be understood by one of ordinary skill in the art that the steps described above may be followed in accordance with a different order without departing from the scope and spirit of the invention disclosed in the present disclosure.

With continued reference to the present disclosure, the invention described herein the present disclosure may include a flare-up prediction algorithm which may include the use of at least one or more the use a machine learning techniques. In accordance with the present disclosure, one embodiment of the present invention, at least one machine learning algorithm may be used predict the likelihood of flare-ups in individuals based on measuring and assessing specific gait measurements and resulting gait data. The present disclosure contemplates the use of at least one on more machine learning technique that may be incorporated into embodiments of the present invention. Furthermore, the present disclosure contemplates the use of at least one or more supervised learning techniques, unsupervised learning techniques, and any combination thereof. It should be understood by one of ordinary skill in the art that the at least one machine learning algorithm may include, but is not limited to: Neural-Networks, Deep Neural Networks (“DNN”), Markov Chain Monte Carlo Neural Networks (“MCMC”), or Bayesian networks. The term “machine learning” should not be construed by one of ordinary skill in the art to be limiting the scope of the invention disclosed in the present disclosure. The terms “machine learning”, “artificial intelligence”, “neural-network”, may all be used interchangeably without departing from the scope and spirit of the invention disclosed in the present disclosure.

In an embodiment, the invention described herein may include a neural network configured to assess when a user is experiencing physical (for example, skeletal, muscular, joint, or back pain) “pain events” based on changes in gait patterns. In an embodiment, the neural network may be configured to target a Unix-based operating system. In one embodiment, the neural network maybe configured in python, and may utilize any modules (for example, pandas, numpy, matplotlib, sklearn, and seaborn) and any database (for example, MongoDB, a cross-platform document-oriented database program, classified as a NoSQL database program, MongoDB uses JSON-like documents with optional schemas). Such a neural network may be configured to run on any platform. As a non-limiting example, the neural network may interact with the AWS EC2 Deep Learning AMI instance. The AWS Deep Learning AMIs may provide machine learning practitioners and researchers with the infrastructure and tools to accelerate deep learning in the cloud, at any scale. One may quickly launch Amazon EC2 instances pre-installed with popular deep learning frameworks and interfaces such as TensorFlow, PyTorch, Apache MXNet, Chainer, Gluon, Horovod, and Keras to train sophisticated, custom AI models, experiment with new algorithms, or to learn new skills and techniques. The invention herein may also utilize Amazon SageMaker for machine learning. Amazon SageMaker or similar platforms may be a fully-managed service that enables developers and data scientists to quickly and easily build, train, and deploy machine learning models at any scale. Such a platform may be configured to remove all the barriers that typically slow down developers who want to use machine learning. However, any number or combinations of languages, modules, databases, and/or platforms may be utilized in building, maintaining, or running the neural network.

The neural network may include any number or combination of algorithms. In an embodiment, an algorithm may be configured to analyze the first set of gait pattern data. For example, accelerometer data and GPS data, derived from the accelerometers and GPS installed in user smart devices, may be captured while individuals are walking. GPS data may determine velocity. In an embodiment, interpolation of initial accelerometer data makes sure that accelerometer data is standardized according to velocity (for example, modification of an accelerometer data so that gait pattern data is isolated from the effect of differing velocities). In another embodiment, an algorithm may be configured to analyze the first set of pain data. In such an algorithm, the same individuals may report their (quantified) subjective experience of pain. In another algorithm, a second set of gait pattern data may be analyzed. In such an algorithm, data is obtained from the same individuals a number of days (for example, X days) after the first set is obtained. In another algorithm, a second set of pain data is analyzed. In such an algorithm, data is obtained from the same individuals a number of days (for example, X days) after the first set was obtained. In a further algorithm, a third set of pain data is analyzed. In such an algorithm, data is obtained from the same individuals a number of days (for example, Y days) after the second set is obtained. In another algorithm, the neural network is configured to predict, based on changes in the user's gait pattern over time, when the user's pain will increase by some metric.

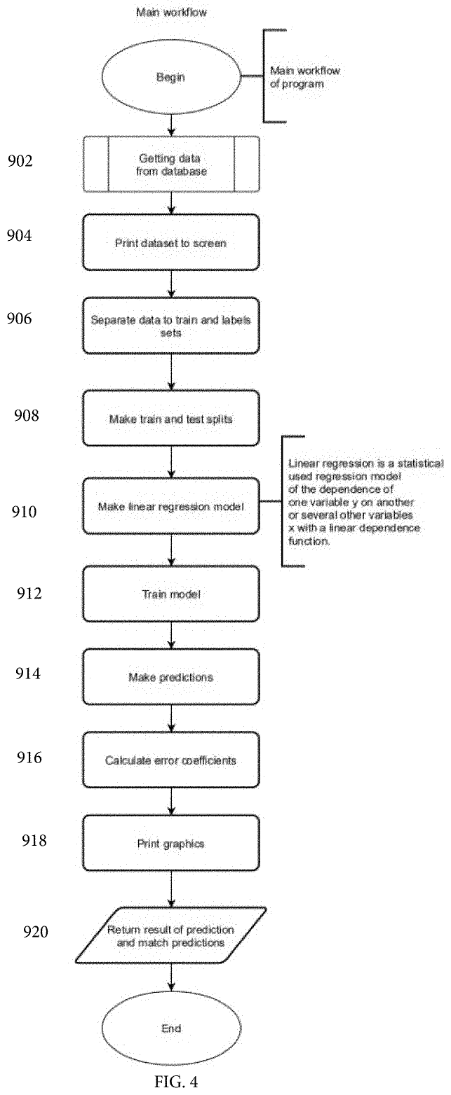

FIG. 4 illustrates a main workflow. In such an embodiment, after the main workflow beings, the system gets data from a database 902 and then prints a dataset to the screen 904. Further, in such an embodiment, the system separates data to train and labels sets 906, making train and test splits 908. Further, the system may then make a linear regression model 910 (for example, the linear regression is a statistical used regression model of the dependence of one variable y on another or several other variables x with a linear dependence function). Next, in the workflow, the system may train a model 912 to make predictions 914. In such a workflow, the system may then calculate error coefficients 916, print graphics 918, and return a result of prediction and match predictions 920.

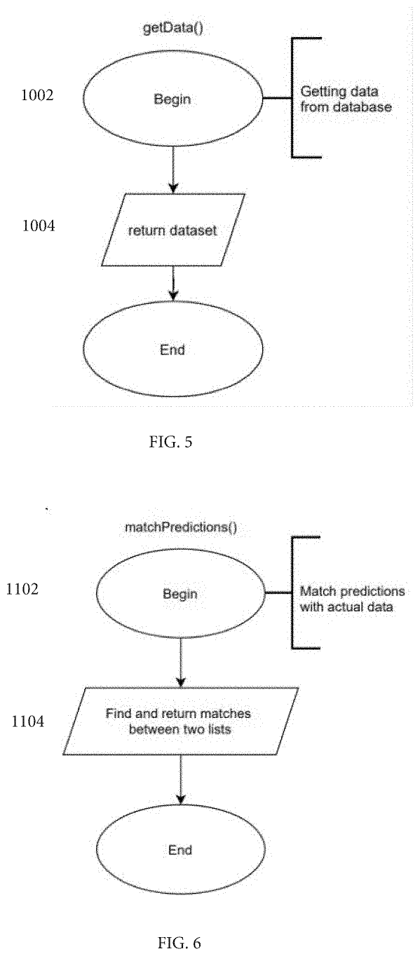

FIG. 5 illustrates a process of getting data from the database. In an embodiment, the system begins to get data from the database 1002 and then returns a dataset 1004. FIG. 6 illustrates a process of searching matches between two lists. In an embodiment, the system begins to match predictions with actual data 1102 and then finds and returns matches between two lists 1104.

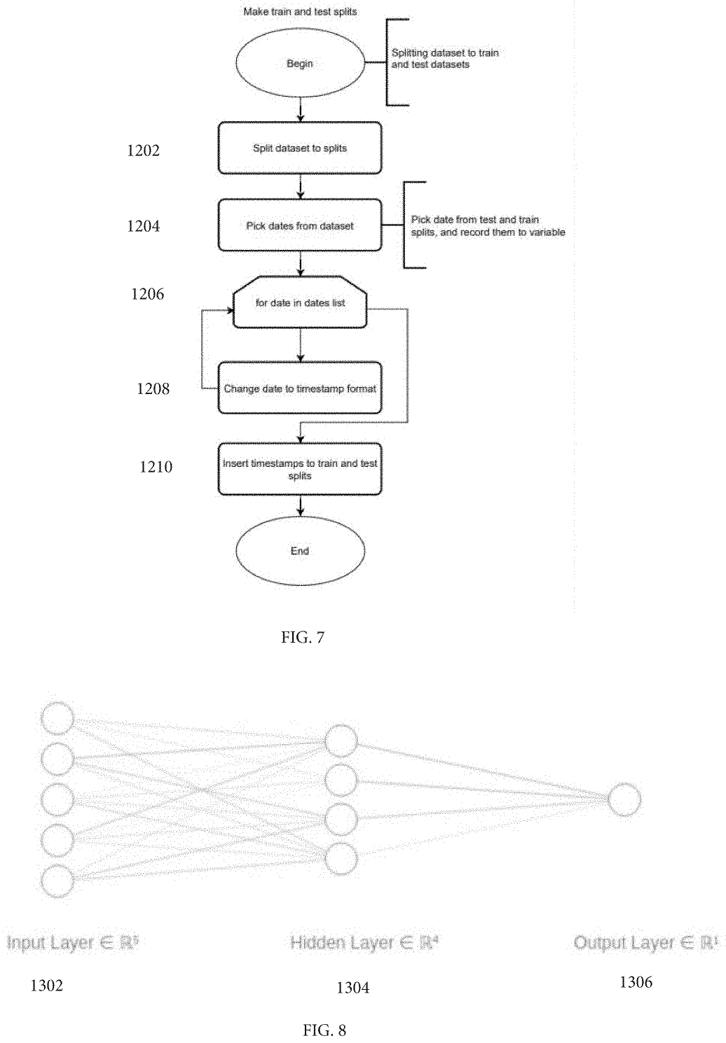

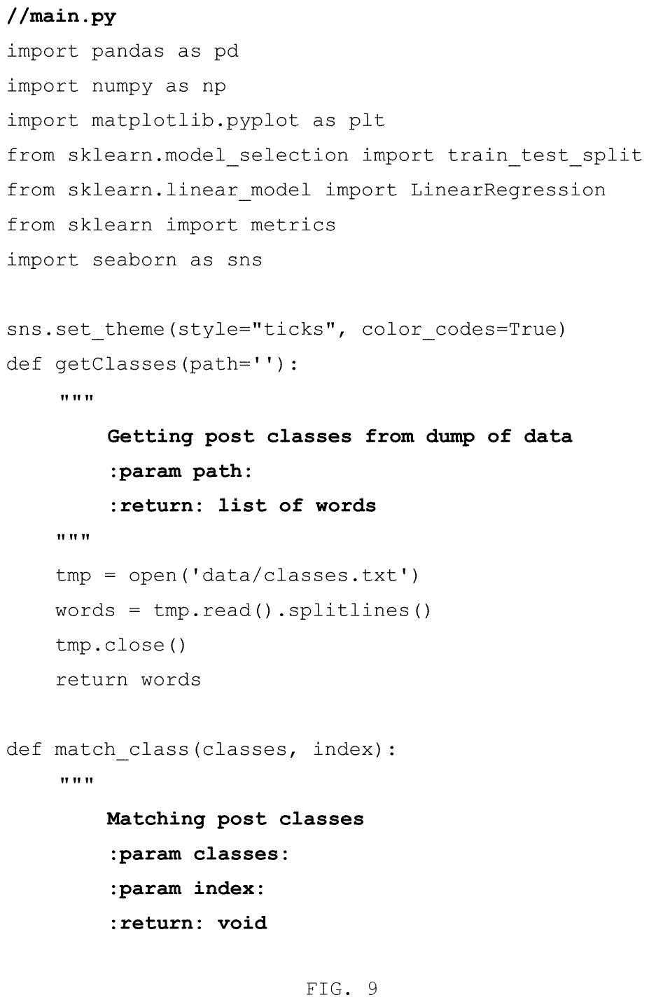

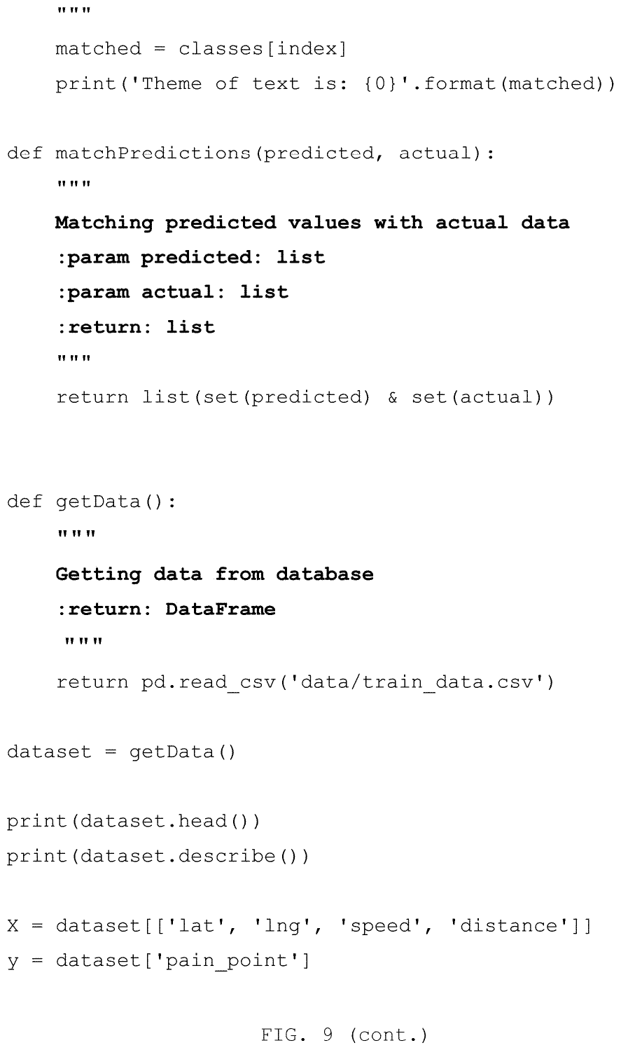

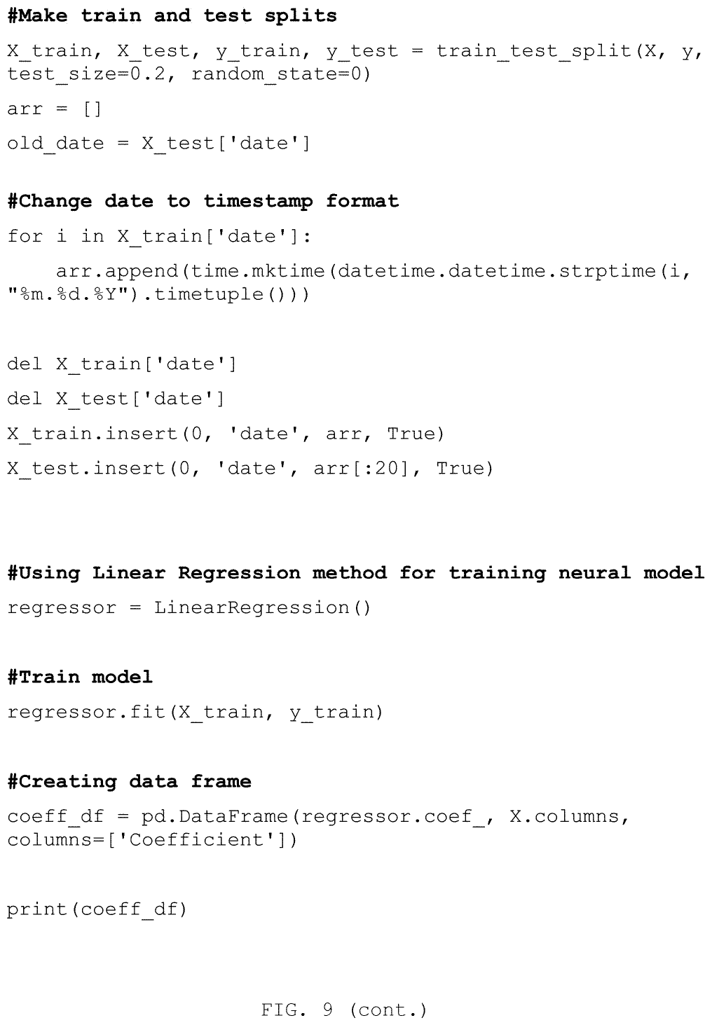

FIG. 7 illustrates a process of splitting global dataset to train and test datasets. In an embodiment, the process may also pick dates from splits and record them to a variable and format it to timestamps. In such an embodiment, the process begins and splits a dataset into splits 1202. Further, the system may then pick dates from the dataset 1204 (for example, picking a date from rest and train splits, and recording it to a variable). The system may then, for a date in the dates list 1206, change the date to a timestamp format 1208 and, for the date in the dates list 1206, insert timestamps to train and test splits 1210.

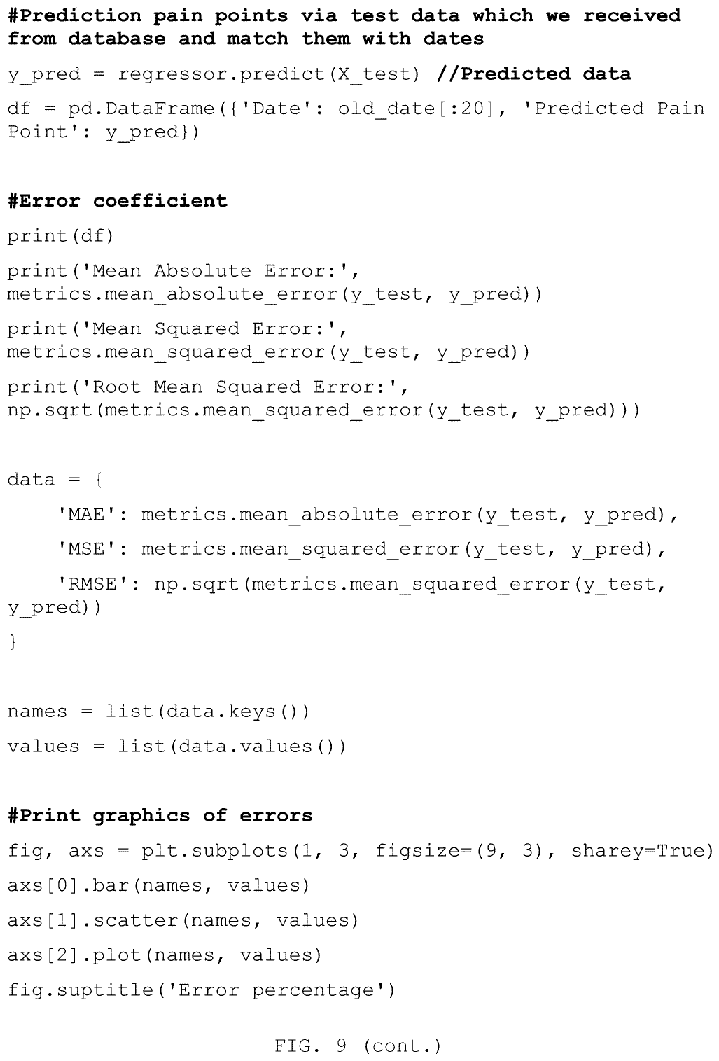

FIG. 8 illustrates a diagram of a neural network. In an embodiment, input layer 1302 consists of parameters such as: date (timestamp converted), latitude, longitude, speed, distance. In an embodiment, information communicates with a hidden layer 1304 before communicating with an output later 1306. The output layer 1306 may be configured to receive pain level points.

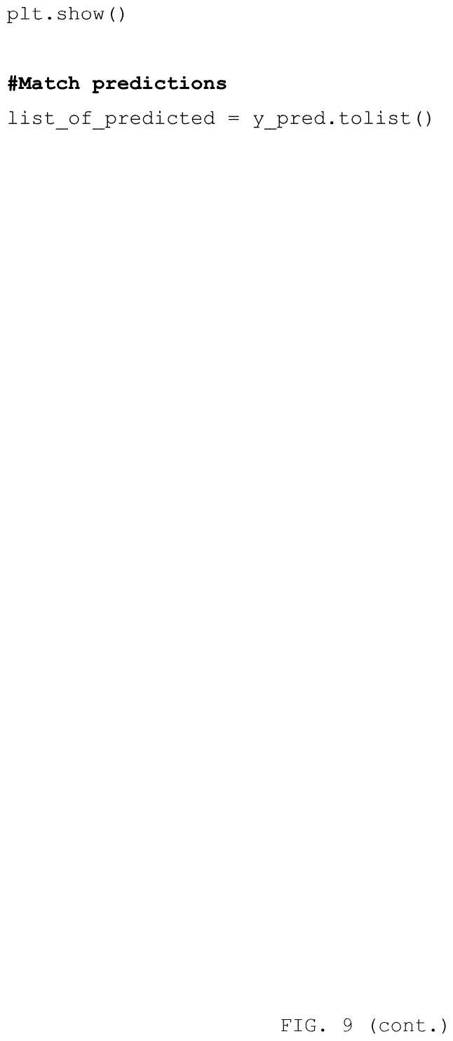

FIG. 9 illustrates computer code configured to execute one or more of the aforementioned algorithms.

In one embodiment, a sample of dataset training structure 1, may appear as follows:

date, lat, lng, speed, distance, pain_point
3.3.2011, 9.9128873226759, 15.006280008241, 4, 4, 7
9.19.2017, 25.60573587362, 29.803164123466, 5, 2, 6
4.17.2016, 24.342020772557, 34.635377665346, 3, 4, 9
6.12.2019, 14.948572992323, 2.8443589307574, 4, 2, 4
10.21.2019, 7.1685942677635, 3.1424513236352, 2, 4, 5
9.9.2014, 33.943060104709, 0.13186739763798, 3, 5, 8
3.4.2013, 14.094471046745, 3.6255557316474, 5, 4, 6
4.1.2017, 14.475663656125, 15.088281683199, 4, 3, 1
6.6.2010, 13.558651773054, 4.1044869842494, 5, 1, 1
. . .

In one embodiment, a sample of dataset training structure 2, may appear as follows:

date, lat, lng, speed, distance, pain_point
6.07.2016, 11.47307787392, 6.8825194434647, 3, 4, 7
1.5.2011, 13.115672727169, 6.5195073888262, 2, 4, 7
6.18.2021, 1.9752700356651, 12.012592521502, 5, 2, 1
2.12.2014, 24.850564722368, 16.54121636252, 2, 5, 9
7.6.2018, 19.457206397996, 21.271846240979, 4, 4, 6
1.19.2013, 18.489117686399, 1.4890508826305, 3, 4, 5
9.15.2021, 9.0868552891011, 10.971630278496, 3, 1, 1
. . .

In one embodiment, a sample of dataset for training structure 3, may appear as follows:

date, lat, lng, speed, distance, pain_point
6.8.2016, 0.48034107707457, 13.539323797235, 2, 1, 7
4.18.2017, 9.0045083500466, 1.4383214867852, 5, 4, 6
4.20.2012, 2.5452043081379, 35.172759391914, 5, 5, 4
3.12.2019, 8.8770679039308, 10.585451557108, 1, 4, 9
8.24.2016, 4.032462893069, 0.67505854399645, 1, 4, 8
9.7.2017, 24.403234941141, 5.8114524305851, 5, 3, 1
. . .

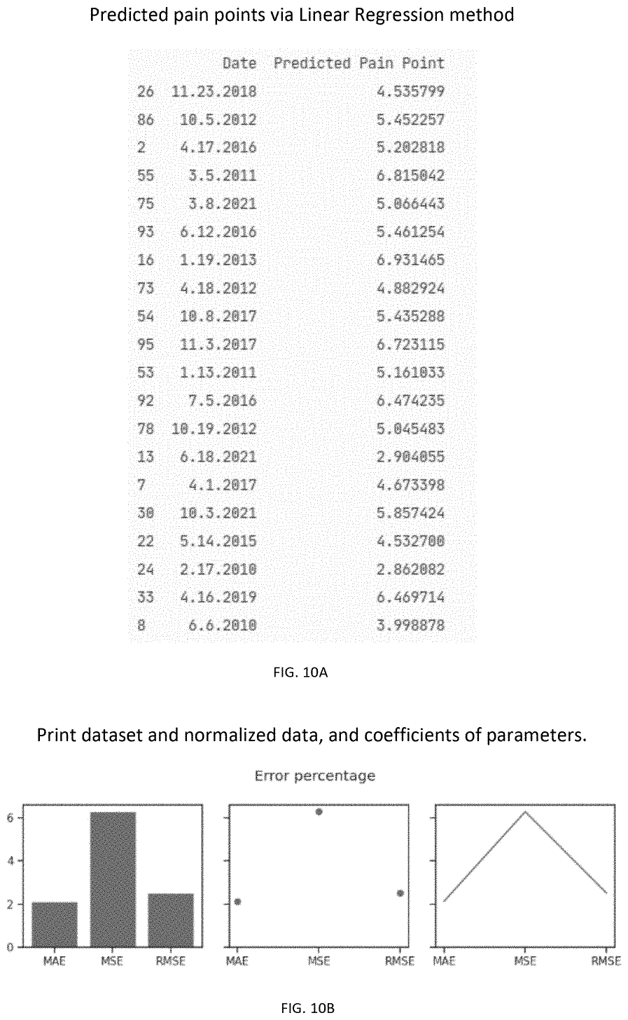

FIG. 10A illustrates an example of a table of predicted pain points via a linear regression method. FIG. 10B illustrates an example of graphs depicting error percentage of predicted pain points.

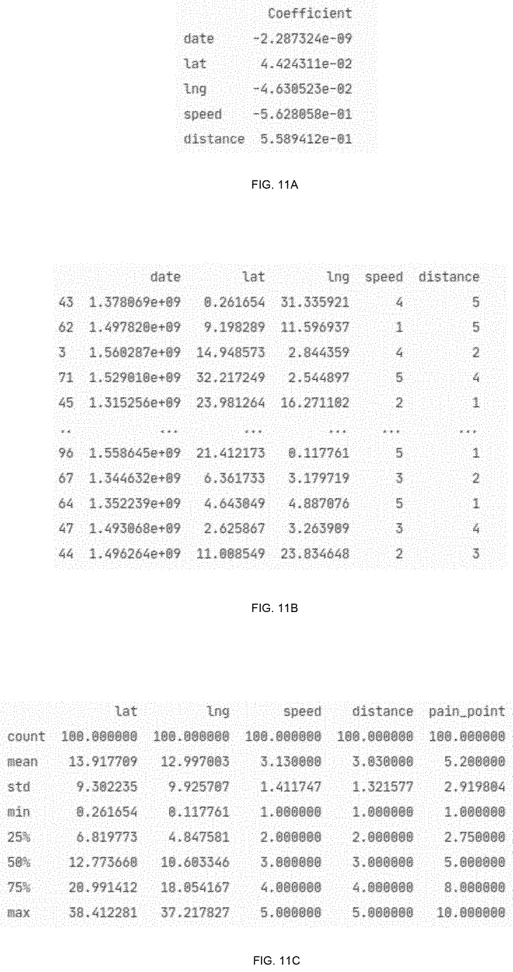

FIGS. 11A-C illustrate tables of printed datasets, normalized data, and coefficients of parameters.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention. Furthermore, the foregoing describes the invention in terms of embodiments foreseen by the inventor for which an enabling description was available, notwithstanding that insubstantial modifications of the invention, not presently foreseen, may nonetheless represent equivalents thereto.

What is claimed is:

1. A system for measuring and assessing biometrically determined changes in gait, the system comprising a processor, a memory, and instructions written on the memory, wherein the instructions when executed by the processor cause the system to:
acquire a baseline gait pattern;
acquire a subsequent gait pattern;
compare the baseline gait pattern to a subsequent gait pattern;
interpolate the baseline gait pattern and the subsequent gait pattern;
validate the baseline gait pattern and the subsequent gait pattern;
correlate the baseline gait pattern and the subsequent gait pattern;
update the baseline gait pattern;
predict a likelihood of a flare-up; and
communicate the likelihood of a flare-up to a user.

2. The system of claim 1 further comprising an accelerometer.

3. The system of claim 1 further comprising a passive infrared sensor.

4. The system of claim 1 wherein the baseline gait pattern is acquired via a mobile device.

5. A method for measuring and assessing biometrically determined changes in gait, the method comprising:
acquiring a baseline gait pattern;
acquiring a subsequent gait pattern;
comparing the baseline gait pattern to the subsequent gait pattern;
interpolating the baseline gait pattern and the subsequent gait pattern;
validating the baseline gait pattern and the subsequent gait pattern;
correlating the baseline gait pattern and the subsequent gait pattern;
updating the baseline gait pattern;
predicting a likelihood of a flare-up;
communicating the likelihood of a flare-up to a user.

6. The method of claim 5 wherein acquiring the baseline gait pattern is accomplished by analyzing an acceleration dataset from a mobile device.

7. The method of claim 5 further comprising the steps of:
storing, via a memory, the baseline gait pattern, the subsequent gait pattern, and the likelihood of flare-up; and
determining a trend on a pre-determined temporal basis.

8. The method of claim 7 further comprising the step of:
evaluating a change to the baseline gate pattern.

9. The method of claim 5 wherein the likelihood of a flare-up is communicated to the user via a mobile device.

10. The method of claim 5 wherein the baseline gait pattern and the subsequent gait pattern are interpolated via linear interpolation.

11. The method of claim 5 wherein the baseline gait pattern and the subsequent gait pattern are interpolated via polynomial spline interpolation.

12. The method of claim 5, further comprising the steps of:
calculating a gait difference score based on obtaining the difference between the baseline gait pattern and the subsequent gait pattern; and
calculating a gait performance score based on the gait difference score.

13. The method of claim 12, further comprising the step of:
analyzing the gait difference score and the gait performance score to determine whether a treatment is effective.
